SEQUENCE LISTING (2 sequences)

SEQ ID NO: 1 - DNA, 1542 bp, Homo sapiens
  CDS (9)..(1490)
agcgcaccatgtctgccctcggagtgaccgtggccctgctggtgtgggcggccttcctcctgctggtgtccatgtggaggcaggtgcacagcagctggaatctgcccccaggccctttcccgcttcccatcatcgggaacctcttccagttggaattgaagaatattcccaagtccttcacccggttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggaatcatttttaataatggacctacctggaaggacatccggcggttttccctgaccaccctccggaactatgggatggggaaacagggcaatgagagccggatccagagggaggcccacttcctgctggaagcactcaggaagacccaaggccagcctttcgaccccaccttcctcatcggctgcgcgccctgcaacgtcatagccgacatcctcttccgcaagcattttgactacaatgatgagaagtttctaaggctgatgtatttgtttaatgagaacttccacctactcagcactccctggctccagctttacaataattttcccagctttctacactacttgcctggaagccacagaaaagccataaaaaatgtggctgaagtaaaagagtatgtgtctgaaagggtgaaggagcaccatcaatctctggaccccaactgtccccgggacctcaccgactgcctgctcgtggaaatggagaaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagttttcaaattgt

SEQ ID NO: 2 - protein, 493 aa, Homo sapiens [sequence omitted]

(19) United States
(12) Patent Application Publication
(10) Pub. No.: US 2002/0183333 A1
(43) Pub. Date: Dec. 5, 2002

(54) Compositions and methods for chemotherapy having reduced nephrotoxicity

(76) Inventors: Sudhir V. Shah, Little Rock, AR (US); Radhakrishna Baliga, Madison, MS (US)

Correspondence Address: GILBRETH & ASSOCIATES, P.C., P.O. BOX 2428, BELLAIRE, TX, 77402-2428, US

(21) Appl. No.: 10/103,520

(22) Filed: Mar. 19, 2002

Related U.S. Application Data
(62) Division of application No. 09/538,518, filed on Mar. 29, 2000.

(51) Int. Cl. A61K 31/497; A61K 31/4525; A61K 31/325; A61K 31/10

(52) U.S. Cl. 514/255.04; 514/320; 514/464; 514/478; 514/365; 514/708

(57) ABSTRACT

Compositions and methods for chemotherapy are disclosed. The compositions and methods disclose herein are effective inhibitors of chemotherapy-induced nephrotoxicity, yet unexpectedly do not inhibit the antineoplastic activity of the chemotherapeutic compound. The chemotherapeutic compositions and methods of the invention comprise an inhibitor of catalytic iron. In a preferred embodiment, the inhibitor is a cytochrome P450 inhibitor and the chemotherapeutic compound is cisplatin.

### BACKGROUND OF THE INVENTION

1. Field of the Invention

The present invention is directed to compositions and methods wherein said compositions and methods comprise an agent that reduces chemotherapy-induced nephrotoxicity without reducing antineoplastic activity of the chemotherapy. The present invention is also directed to compositions and methods for chemotherapy comprising an agent selected from the group consisting of iron chelators, hydroxy radical scavengers and cytochrome P450 inhibitor, wherein said agent reduces chemotherapy-induced nephrotoxicity without reducing antineoplastic activity of the chemotherapy.

2. Description of the Related Art

Cisplatin (CP) is one of the most effective chemotherapeutic agents used in the treatment of a variety of human solid tumors (Rozencweig et al., 1997, Ann.Intern.Med.86:803-812). Unfortunately the efficacy of cisplatin is limited by the concomitant cisplatin-induced nephrotoxicity that develops at the S₃ segment of the proximal tubule (Balchley and Hill, 1981, Ann.Intern.Med.95:628-632). Reactive oxygen metabolites (ROMs) have been implicated as the mediators of this renal injury (Sugihara and Gemba, 1986, Jpn.J.Pharmacol.40:353-355).

Catalytic iron is believed to play an important role in the generation of ROMs via the metal-catalyzed Haber-Weiss reaction, and/or in the generation of the highly reactive iron-oxygen complexes such as ferryl or perferryl ions (Halliwell and Gutteridge, 1990, Meth.Enzym.186:1-85).

In vivo most of the iron found is bound to heme and non-heme proteins and does not directly catalyze the formation of hydroxyl radicals (Halliwell and Gutteridge, 1990, Meth.Enzym.186:1-85). However, hydrogen peroxide and organic hydroperoxides can oxidatively degrade hemoglobin thereby resulting in the release of iron from heme and enabling the iron to function in catalyzing ROM generation (Gutteridge, 1986, FEBS Letters 201(2): 291-295).

Attempts at maximizing the therapeutic benefits of chemotherapeutic agents, such as cisplatin, while decreasing chemotherapy-induced nephrotoxicity have been disclosed

For example, the combination of cimetidine and verpamil has been tested in a limited number of patients treated with cisplatin with beneficial effects on renal hemodynamics and renal function (Sleijfer et al., 1987, Cancer 60(11): 2823-2828). Verapamil by itself has been shown to have no effect in a rat model of cisplatin induced nephrotoxicity (Capasso et al., 1990, Clin. Nephrol. 33:184-191). The iron chelator dexrazoxane has been shown to decrease the frequency and severity of cardiotoxicity induced by doxorubicin in women with breast cancer (Speyer et al., 1992, J. Clin. Oncol. 19(1): 117-127). Donfrancesco et al., 1992, Am. J. Clin. Oncol. 15(4): 319-322, combined desferoxamine with chemotherapy for the treatment of neuroblastoma with beneficial results. Multicenter clinical trials are underway looking at the safety and efficacy of amifostine to prevent myelotoxicity of high dose carboplatin with promising results (Borsi et al., 1997, Proc. Am. Soc. Clin. Oncol.16:1400).

WO9749427, issued Dec. 31, 1997, to Kawauchi et al, discloses administration of antibody inhibitory to type II phospholipase A2 in order to ameliorate cisplatin-induced renal disorders.

WO9840095, issued Sep. 17, 1998, to Kadomatsu et al, discloses agents containing proteins belonging to the midkine family for relieving nephropathy induced by antineoplastic agents such as, cisplatin, or acute hepatitis.

Baliga et al., 1998, Kidney International 53:394-401, disclose a hypothesis suggesting a role for iron in mediating tissue injury via hydroxy radicals in a cisplatin-induced model of nephrotoxicity.

Baliga et al., November 1998, Kidney International 54(5), 1562-1569, disclose a role for cytochrome P450 as a source of catalytic iron in cisplatin-induced nephrotoxicity.

WO9905299, issued Feb. 04, 1999, to Waxman et al, discloses killing of neoplastic cells by use of NADPH-cytochrome P450 reductase gene transfer in combination with cytochrome P450 gene transfer to enhance sensitivity of tumor cells to anti-cancer drugs.

Despite the advances made in the art, there remains a need for compositions and methods for highly effective chemotherapy comprising an agent that reduces chemotherapy-induced nephrotoxicty without inhibiting the antineoplastic effects of a chemotherapeutic compound. There also remains a need for compositions and methods for chemotherapy comprising cisplatin, and an agent that inhibits catalytic iron, such as, an iron chelator, a hydroxy radical scavenger or a cytochrome P450 inhibitor, wherein the agent reduces chemotherapy-induced nephrotoxicty without inhibiting antineoplastic effects of a chemotherapeutic compound. There also remains a need for compositions and methods for chemotherapy comprising cisplatin and an agent that inhibits catalytic iron, such as, an iron chelator, a hydroxy radical scavenger or a cytochrome P450 inhibitor, wherein the agent reduces chemotherapy-induced nephrotoxicty without inhibiting antineoplastic effects of cisplatin.

### SUMMARY OF THE INVENTION

It is an object of the present invention to provide compositions and methods for chemotherapy useful for treating a neoplasia, without inducing nephrotoxicity.

It is another object of the present invention to provide compositions and methods for highly effective chemotherapy comprising an agent which reduces chemotherapy-induced nephrotoxicity without affecting the antineoplastic efficacy of the chemotherapeutic compound.

It is even another object of the present invention to provide compositions and methods for chemotherapy comprising cisplatin and an agent that inhibits catalytic iron, such as, an iron chelator, a hydroxy radical scavenger or a cytochrome P450 inhibitor, wherein chemotherapy is highly effective and wherein the agent reduces chemotherapy-induced nephrotoxicity without affecting the antineoplastic efficacy of the chemotherapeutic compound.

All references cited herein, including all research articles, U.S. and foreign patents and patent applications, are specifically and entirely incorporated by reference.

### BRIEF DESCRIPTION OF THE DRAWINGS

FIG. 1A shows the concentration-dependent effects of cisplatin antineoplastic activity in LLC-WRC 256 cells. Confluent LLC-WRC 256 cell monolayer was incubated with various concentrations of cisplatin as indicated in the figure. Trypan blue exclusion assay was performed at the end of incubation.

FIG. 1B shows the time-dependent effects of cisplatin antineoplastic activity in LLC-WRC 256 cells. Confluent LLC-WRC 256 cell monolayer were incubated with a cisplatin concentration of 500 μg/ml, for the times indicated in FIG. 1A. Trypan blue exclusion assay was performed at the end of incubation.

FIG. 2 shows the effects of an iron chelator and of a hydroxyl radical scavenger on the antineoplastic efficacy of cisplatin as measured by trypan blue exclusion. Confluent LLC-WRC 256 cells were preincubated with an iron chelator, deferoxamine (DFO, 1 mM), and a hydroxyl radical scavenger, dimethyl sulfoxide (DMSO, 5 mM) for 30 minutes prior to the addition of cisplatin. (500 μg/ml for 5 hours). LLC-WRC 256 cells were also incubated with HBSS, DFO (1 mM) or DMSO (5 mM) for 5 hours as controls. Values shown are means±SE, n=5, *p<0.01, compared with control cells.

FIG. 3 shows the effects of general CYP inhibitors on the antineoplastic efficacy of cisplatin as measured by trypan blue exclusion. Confluent LLC-WRC 256 cell monolayer was preincubated with the general CYP inhibitors, cimetidine (CM, 2 mM) and piperonyl butoxide (PB, 1 mM) for 30 minutes prior to the addition of cisplatin (500 μg/ml for 5 hours). LLC-WRC 256 cells were also incubated for 5 hours with HBSS, CM (2 mM) or PB (1 mM) as controls. Values shown are means±SE, n=5, *p<0.01, compared with control cells.

### DETAILED DESCRIPTION OF THE INVENTION

Iron has been shown to be important in cytotoxicity in a variety of models of tissue injury, including injury induced by therapeutic agents, such as, for example cyclosporin, gentamicin and cisplatin (Halliwell and Gutteridge, 1990, Meth. Enzym 186:1-85; Baliga et al, 1999, Drug Metabolism Reviews 31(4), 971-998). Iron chelators have been shown to reduce cisplatin-induced nephrotoxicity.

Based on the above data and additional data from a number of systems, one of skill in the art would anticipate that iron chelators would also reduce cisplatin-induced cytotoxicty in neoplastic cells. Thus, one of skill in the art would not expect such agents to be useful in chemotherapy since the agents are expected to inhibit cisplatin-induced cytotoxicity, and thereby would reduce the efficacy of cisplatin as an antineoplastic agent.

Significantly, the applicants have unexpectedly discovered that the agents of the invention, while functioning as efficient inhibitors of chemotherapy-induced nephrotoxicity, do not inhibit antineoplastic activity of a chemotherapeutic compound. Specifically, the inventors have found that inhibitor agents according to the invention effectively inhibit cisplatin-induced nephrotoxicity yet do not affect tumoricidal activity of cisplatin.

The present invention is directed to compositions and methods for treating an individual having a cancer. As used herein, the term “cancer” encompasses any cell or cells whose normal growth control mechanisms are disrupted, thereby providing the potential for uncontrolled cell proliferation. The term cancer includes both benign and malignant neoplastic cells/tumors in both the central nervous system and the periphery, wherein periphery is intended to mean all other parts of the body outside of the brain or spinal cord.

The compositions and methods of the present invention are suitable for treating an individual afflicted with any neoplasia/cancer. The neoplastic cells killed by the compositions and methods of the present invention include cells of tumors, neoplasms, carcinoma, sarcomas, papillomas, leukemia, lymphomas and the like. Of general interest are solid tumors, preferably any solid tumor that may be treated with cisplatin chemotherapy.

The compositions and methods of the present invention provide for highly effective chemotherapy, that does not result in nephrotoxicity. In one embodiment of the present invention, compositions and methods comprise an agent that inhibits the available source of catalytic iron at the site of cytotoxicity, for example, the kidney, while not affecting the antineoplastic activity of a chemotherapeutic compound.

As used herein, the phrase “chemotherapeutic compound” includes any chemotherapeutic agent known in the art, including, but not limited to, cisplatin, isosfamide, 5-FU, carboplatin, ibuprofen and diclofenac. Generally the chemotherapeutic compounds useful in the present invention are cisplatin, isosfamide, 5-FU, carboplatin, more preferably cisplatin, 5-FU, or isosfamide, and even more preferably the chemotherapeutic compound is cisplatin.

Administration of chemotherapy is dependent on at least, the chemotherapeutic compound being administered. Other factors include, the status of the patient, and the status of the neoplasia of the patient. Administration and optimization of chemotherapeutic compounds is standard and known in the art.

The chemotherapeutic compounds of the invention may function in killing cells through numerous mechanisms. Without being limited by theory, the principle mechanism of the antitumor activity of CP is believed to be the formation of intrastrain and interstrain cross-links of the DNA helix which in turn block DNA replication (Weiss and Christin, 1993, Drug, 46:360-377.).

The present invention is directed to the use of a chemotherapeutic compound to kill cancer cells, in. addition to the use of an inhibitor agent to reduce toxicity associated with the chemotherapeutic compound. Generally, the toxicity associated with the chemotherapeutic compounds of the invention is nephrotoxicity and/or acute renal failure.

The chemotherapeutic compound and the inhibitor agent of the present invention may be administered together, or they may be administered sequentially in any order For example, the individual may also receive the inhibitor agent together with the chemotherapeutic compound. It is also possible that the individual to be treated may receive an effective dose of the inhibitor agent prior to receiving an effective dose of the chemotherapeutic compound. It is also possible that the individual to be treated is first administered a therapeutic dose of a chemotherapeutic compound, followed by an effective dosage of the inhibitor agent. In a preferred embodiment, an effective dose of the inhibitor agent is administered concurrently with the effective dose of the chemotherapeutic compound. Administration and dose, according to the present invention, are discussed below.

Another embodiment of the present invention is directed to compositions and methods for chemotherapy comprising a composition comprising an inhibitor agent that reduces the ability of a cytochrome P450 superfamily member to serve as a source of catalytic iron. This may comprise partially reducing or completely reducing the ability of a CYP to serve as a source of catalytic iron. Preferably, the inhibitor reduces the ability of a CYP to serve as a source of catalytic iron.

As used herein, the phrase “cytochrome P450” (CYP) is intended to include any member/isoenzyme of the CYP superfamily, which includes the mammalian CYP1, CYP2, CYP3 and CYP4 families (Omiecinski, et al., 1999, Toxicological Sciences 48:151-156). Thus, CYP includes the mammalian cytochrome P450 genes and their gene products, such as, P450 1A1, 1A2, 1B1, 2B1, 2B2, 2B4, 2B5, 2B6, 2B11, 2A6, 2C6, 2C8, 2C9, 2C11, 2C18, 2C19, 2D6, 2E1, 3A4, 3A5, 3A7, 4A1 and 4B1. Of particular interest is the CYP isozyme CYP2E1, which is in renal tubules. A complete mRNA sequence and translation sequence of a human CYP2E1, GenBank accession number AF182276, is provided herein as SEQ.ID.NO.: 1. The phrase “cytochrome P450” (CYP) also includes allelic variants, site-specific mutants, and chimeric constructs of members of the CYP superfamily (Chang et al., 1997, Pharmacogenetics 7:211-221; Szklarz et al., 1995, Biochemistry 34:14312-14322; He et al, 1997, Biochemistry 36:8831-8839).

There are numerous CYP inhibitors/agents useful in the present invention for reducing the ability of CYP to serve as a source of catalytic iron, all of which are included in the present invention.

As used herein the phrase “CYP inhibitor” includes any agent that reduces the ability of a CYP to serve as a source of catalytic iron. According to the present invention, CYP inhibitors include agents that directly interact with a CYP, indirectly interact with a CYP, directly or indirectly reduce the expression of a CYP, function in a biochemical pathway upstream of a CYP, and agents that function in a biochemical pathway downstream of a CYP. The interaction between the CYP and the CYP inhibitor may be a direct or an indirect interaction. The inhibitors useful in the present invention may function by interacting with the heme iron of a CYP, thereby blocking or reducing the availability of the iron to serve as a catalytic iron source. In addition, the inhibitor agents of the invention may inhibit CYP at the level of gene expression, gene transcription, translation, or CYP function.

Non-limiting examples of agents suitable for use in the present invention as CYP inhibitors include, cimetidine (CM), piperonyl butoxide (PB), safrole, isosafrole, myristicinfurfylline, diethyldithiocarbamate, chlormethiazol, piperine, disulfiram, diallyl sulfid, malotilate, allylmercaptan, methylprazole, orphenadrine, arylacetylenes, clorgyline and diphenhydramine. Generally the CYP inhibitors useful in the present invention are selected from the group consisting of cimetidine, piperonyl butoxide, piperine, diethyldithiocarbamate, chlormethiazol, disulfiram and diallyl sulfid. Preferably, the CYP inhibitor used in the present invention is cimetidine, piperonyl butoxide, piperine, diethyldithiocarbamate, and chlormethiazol.

Without being limited by theory, it is believed cimetidine has imidazole and cyano groups that inhibit CYP by interacting with the heme iron (Rendic et al., 1983, Drug Metab. Dispos. 11:137-142). This inhibitory effect of cimetidine (CM) is specific for CYP, as CM does not interact with other heme enzymes (Baird et al., 1987, Biochem Pharmacol. 36:4366-4369). Another CYP inhibitor of the invention, piperonyl butoxide (PB), is believed to yield a metabolite that binds to the heme moiety of CYP and inhibits microsome metabolism (Mays et al., 1989, Biochem.Pharmacol 38:1647-1655). The inhibitors of the invention may function in an inhibitor-specific manner to inhibit CYP. The mechanisms provided for CM and PB are examples of at least two different mechanisms by which CYP inhibitors may function.

Additional agents useful as inhibitors in the compositions and methods of the present invention include agents that inhibit reactive oxygen metabolites (ROMs).

Oxygen usually accepts four electrons and is converted directly into water. However, partial reduction of oxygen also occurs in biological systems, leading to the generation of partially reduced and potentially toxic ROMs. For example, when oxygen accepts one electron, superoxide anion, a free radical, is generated. When oxygen accepts two electrons either directly, or by the dismutation of superoxide, hydrogen peroxide is formed. Further reduction of oxygen leads to the generation of the highly reactive hydroxyl radical. Inhibition of ROMs according to the present invention, may be by inhibiting at least one step of a biochemical pathway leading to the formation of ROMs. As used herein, ROM is intended to include any of the reactive oxygen metabolites such as, for example, free-radical species, superoxide radicals, hydroxyl radicals, and other oxygen metabolites such as, hydrogen peroxide and hypochlorous acid.

Without being limited by theory, it is possible that an increase in the generation of hydrogen peroxide may result in direct oxidative attack on the heme moiety of CYP promoting the heme destruction and the release of iron. One mechanism by which inhibitors of the present invention may function is to inhibit this release of iron.

Additional inhibitor agents useful in the present invention include agents that inhibit catalytic iron, including, for example, iron chelators, and hydroxy radical scavengers. One of the mechanisms by which iron mediates tissue injury is the generation of hydroxyl radicals via the iron catalyzed Haber-Weiss reaction. Some of the inhibitors of the invention function to inhibit or reduce this hydroxyl radical generation.

Iron chelators suitable for use in the present invention, include, for example, deferoxamine, deferiprone, HBED, PIH, 1,10-phenanthroline. Hydroxy radical scavengers suitable for the present invention include agents such as DMSO, mannitol, benzoic acid, and dimethylthiourea (DMTU), all of which are suitable inhibitors for the compositions and methods of the present invention.

The compositions and methods of the present invention may comprise a single CYP inhibitor, or any combination of the CYP inhibitors disclosed herein.

Additional inhibitors suitable for use in the compositions and methods of the invention include agents wherein the agent is a DNA, cDNA, RNA or polypeptide sequence. Suitable examples of such agents include, an antisense CYP sequence which inhibits transcription or translation of a CYP, transcription factors which decrease expression of a CYP gene, factors which affect translation of a CYP mRNA, factors which decrease the stability/half-life of a CYP mRNA molecule, factors which decrease the stability/half-life of a CYP polypeptide, and factors which interact with a CYP polypeptide, such as an antibody which specifically interacts with an epitope of a CYP. The material and methods for manipulating and producing these types DNA, cDNA, RNA and polypeptides are known in the art.

Expression vectors comprising a sequence inhibitory to transcription of a CYP gene or comprising a sequence inhibitory to translation of a CYP mRNA are within the scope of the CYP inhibitors defined herein. Expression vectors suitable for the present invention may comprise an antisense CYP sequence, or a sequence encoding a negative regulator of transcription of a CYP gene.

Preferably the vectors of the invention comprise a CYP antisense sequence which will inhibit the mRNA of a CYP gene. Generally, preferred CYP targets for antisense technology are the CYP2 family members, more preferred are the CYP2E sequences, and even more preferred is the CYP2El sequence.

Vectors comprising a DNA sequence which encodes a polypeptide of a CYP dominant negative mutant, or a vetor comprising a DNA sequence encoding a CYP mutant may also be useful inhibitors in the present invention.

Suitable vectors are known in the art and include, for example, mammalian expression vectors and viral vectors. Examples of viral vectors suitable for use in the present invention include: retroviruses; adenoviruses; adenoviral/retroviral chimeras; adeno-associated viruses; herpes simples virus I or II; parvovirus; and reticuloendotheliosis virus. Other possible viral vectors may be derived from poliovirus, papillomavirus, vaccinia virus, lentivirus, as well as chineric vetors incorportation favorable aspects of any two or more of the the above viruses.

For guidance in the construction of gene therapy vectors and in the introduction thereof into affected individuals for therapeutic purposes, WO 99/05299 cites as well as U.S. Pat. Nos. 5,631,236; 5,688,773; 5,691,177; 5,670,488; 5,601,818; and WO 95/06486.

Generally, methods are known in the art for viral infection of the cells of interest. The virus can be injected into a patient bearing a neoplasm, either at, into, or near the cells interest. Preferably, the treatment of the present invention will be by infection of cells of the kidney.

The compositions of the present invention may further comprise a pharmaceutically acceptable carrier/vehicle. Pharmaceutically acceptable carriers/vehicles are known in the art and include aqueous solutions, non-toxic excipients, including salts, preservatives, buffers and the like, propylene glycol, polyethylene glycol, lipids, liposomes, vegetable oil, injectable organic esters such as ethyloleate, water, saline solutions, parenteral vehicles such as sodium chloride and Ringer's dextrose, glycerol, lipids, alcohols.

The chemotherapeutic compositions as well as the inhibitor compositions of the present invention may be in any form known in the art, such as an orally digestible form, a sterile injectable form, forms suitable for delayed release, and forms that are enterically coated. Compositions of the invention may be in solid forms, including, for example, powders, tablets, pills, granules, capsules, sachets and suppositories, or may be in liquid forms including solutions, suspensions, gels and emulsions.

Administration of the compositions of the present invention to a recipient may be by any method known in the art. Thus, administration of the present invention to a recipient may be by a route selected from oral, parenteral (including, subcutaneous, intradermal, intramuscular, and intravenous) and rectal. For increased efficacy, the compositions of the present invention may be administered via localized delivery to the cancer. Generally the chemotherapeutic compound is administered to a recipient orally or parenterally, and the composition of the present invention comprising an inhibitor agent of the present invention is administered orally or intravenously. Preferably, the chemotherapeutic compound is cisplatin, which is administered orally or intravenously, and the inhibitor agent is cimetidine, which is administered orally or intravenously.

The compositions and methods of the present invention may be administered to a recipient/patient as a single dose unit, or may be administered in several dose units, for a period ranging from one day to several years. The dose schedule is dependent upon at least the severity of the cancer, as well as the mode of administration. The effective dose of the compositions of the present invention is dependent upon the body weight (BW) of the recipient/patient and also upon the chosen method of administration.

Generally, the compositions of the present invention are administered for a time period ranging from about one day to about two years, preferably from about three days to about one year, even more preferably from about one week to about ten months, still more preferably from about ten days to about eight months, yet more preferably from about two weeks to about six months, and even still more preferably from about two and a half weeks to about three months. In a particularly preferred embodiment, administration of the compositions of the invention correlates with administration of a chemotherapeutic compound.

Administration of a chemotherapeutic compound is dependent upon at least, the status of the patient, the status of the neoplasia of the patient. Optimizing and administering chemotherapy is known in the art. Generally the chemotherapeutic compound of the present invention is administered intravenously at a concentration of 50-100 mg/m², once every 3-4 weeks.

Preferably the inhibitor is selected from the group consisting of cimetidine, piperine, DEDC and chlormethiazole. Generally, cimetidine is administered via an oral or intravenous route at a concentration in the range of about 10 to about 50 mg per kilogram of body weight, preferably about 15 to about 45 mg per kilogram of body weight, and more preferably about 20 to about 40. Generally cimetidine is given every six hours in a twenty four hour period.

Generally piperine is administered by an oral or intravenous route in an amount ranging from about 100 to about 300 mg, preferably from about 125 to about 250 mg, and more preferably from about 150 to about 200 mg. Generally piperine is administered orally once every twenty-four hours.

The inhibitor DEDC is generally administered orally or intravenously at a concentration ranging from about. 0.25 to about 2.5 g/m², preferably from about 0.4 to about 2.0 g/m², even more preferably from about 0.5 to about 1.8 g/m², and still more preferably from about 0.6 to about 1.6 g/m². Generally, DEDC is given once a day.

The inhibitor chlormethiazole is generally administered orally in an amount ranging from about 0.5 to about 3.5 g, preferably from about 0.75 to about 3.0 grams even more preferably from about 1.0 to about 2.75 grams, and still more preferably from about 1.2 to about 2.4 grams. Generally chlormethiazole is given once every twenty-four hours.

Typically the expression vectors of the invention would be prepared as an injectable, either as liquid solutions or suspensions; solid forms suitable for solution in, or suspension in, liquid prior to injection may also be prepared. The preparation may also be emulsified.

In general, the virus in provided in a therapeutically effective amount to infect target cells. The quantitiy of the viral vector to be administered, both according to number of treatments and amount, will also depend on factors such as the clinical status, age, and weight of the patient to be treated. Generally, viral vectors of the invention are administered in titers ranging from about 1×10⁵ to about 1×10⁹ cfu per ml, although ranges may vary.

The compositions and methods of the present invention are suitable for any individual afflicted with a cancer. Suitable individuals include mammals such as, humans, dogs, cats, horses, cows, sheep, goats, pigs, rats and mice. The compositions and methods of the present invention are also suitable for use in any tissue or cells, including that of kidney, spinal cord, brain, liver, lung, intestine and skin. Thus the present invention is useful to medical and health care professionals including, medical doctors, and veterinarians, as well as research scientists.

Another embodiment of the present invention is directed to method for treating a patient. Generally the method comprises the steps of administering a therapeutically effective amount of a first composition to a patient, wherein said patient has a neoplasia comprising neoplastic cells, and wherein said first composition comprises an inhibitor of catalytic iron which reduces chemotherapy-induced nephrotoxicity without reducing the antineoplastic activity of a chemotherapeutic compound; and administering a therapeutically effective amount of a second composition to the patient, wherein said second composition comprises a chemotherapeutic compound.

Generally, step (a) and step (b) of the method are performed at the same time. Preferably, the first and second composition are given as a single dose concurrently. The first and second compositions may also be combined into a third composition administered to a patient as a single dose.

Alternately, step (a) and step (b) of the method may be separated by a period of time. Generally, the period of time separating step (a) and step (b) ranges from about half a minute to about one week, preferably from about one minute to about 24 hours, more preferably from about two minutes to about 12 hours, even more preferably from about five minutes to about 6 hours, still more preferably from about 10 minutes to about 5 hours and yet more preferably from about 15 minutes to about 4 hours. The mode of administering the first composition may be oral, parenteral or rectal, and the mode of administering the second composition may be oral, parenteral or rectal. If administered separately, the compositions may or may not be administered by similar modes.

Another method of the invention is directed to a method for treating a patient by administering a therapeutically effective amount of a first composition to a patient having a neoplasia, wherein said first composition comprises an inhibitor of catalytic iron selected from the group consisting of cimetidine, piperonyl butoxide, piperine, diethyldithiocarbamate, clormethiazol, deferoxamine, dimethyl sulfoxide, and any combination thereof. The method may further comprising the step of administering a therapeutically effective amount of a second composition to the patient, wherein said second composition comprises a chemotherapeutic compound, such as, cisplatin. Preferably step (a) and step (b) are performed concurrently.

Another method of the invention is directed to a method for treating a patient by administering a therapeutically effective amount of a first composition to a patient having a neoplasia, wherein said first composition comprises an agent that inhibits the ability of a cytochrome P450 2E family member to serve as a source of catalytic iron. The method may further comprising the step of administering a therapeutically effective amount of a second composition to the patient, wherein said second composition comprises a chemotherapeutic compound. Preferably the agent is selected from the group consisting of cimetidine, piperonyl butoxide, piperine, diethyldithiocarbamate, clormethiazol, deferoxamine, dimethyl sulfoxide, and any combination thereof, and the chemotherapeutic compound is cisplatin.

Another method of the invention is directed to a method for treating a patient by infecting a kidney of a patient with a vector comprising a sequence encoding an inhibitor of a cytochrome P450. The method may further comprising the step of administering a therapeutically effective amount of a composition comprising a chemotherapeutic compound, such as cisplatin, to the patient. Generally, the inhibitor is an antisense cytochrome P450 sequence, preferably the sequence is a partial sequence of a cytochrome P450 2E family member. Step (a) and (b) may be carried out concurrently, or alternatively, they may be separated by a period of time.

Another method of the invention is directed to a method for treating a patient by infecting a kidney of a. patient with a vector comprising a sequence encoding an inhibitor of a source of catalytic iron, such as, a cytochrmoe P450 superfamily member. The method may further comprising the step of administering a therapeutically effective amount of a composition comprising a chemotherapeutic compound, such as cisplatin, to the patient. Generally, the sequence is an antisense cytochrome P450 sequence, preferably the cytochrome P450 sequence SEQ.ID.NO.: 1.

The effects of a chemotherapeutic compound, such as CP, may be examined by performing a time course study and testing dose-dependent effects of the chemotherapeutic compound on irreversible cell death in cells, such as, for example LLC-WRC 256 cells. The trypan blue exclusion assay is a useful assay for measuring viability.

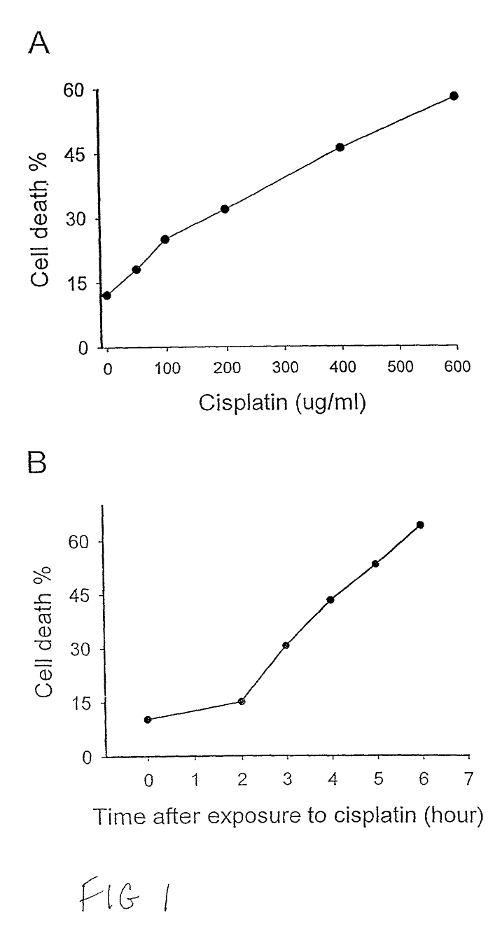

Referring now to FIG. 1A there is shown the antineoplastic activity of different concentrations of cisplatin on a tubule cell line, LLC-WRC 256 cells. Treatment of cells with different concentrations of CP, ranging from 0-600 μg/ml, is carried out for a 4 hour incubation. As shown, significant cell death in LLC-WRC 256 cells, occurs at a low dose of CP, 50 μg/ml, and cytotoxicity increase with higher doses, for example, 100-600 μg/ml. Based on the concentration dependent effects of CP, the concentration of 500 μg/ml is an example of a CP dose required to produce submaximal injury.

Referring now to FIG. 1B, there is shown a time course analysis of the antineoplastic activity of cisplatin at a concentration of 500 μg/ml, for a time period of 0-6 hours. Exposure of LLC-WRC 256 cells to CP at a dose of 500 μg/ml results in significant cell death at 5 hours.

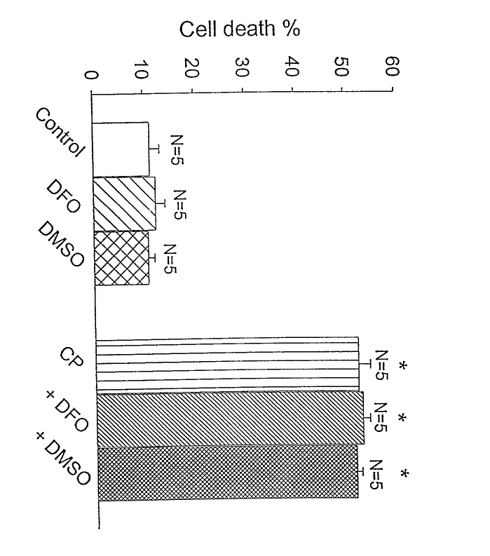

Referring now to FIG. 2 there are shown the effects of two different inhibitor agents of the invention. Neither an iron chelator, nor a hydroxyl radical scavenger of the invention inhibit the antineoplastic efficacy of cisplatin as measured by trypan blue exclusion. According to the invention, confluent LLC-WRC 256 cells are preincubated with the iron chelator deferoxamine (DFO) at a concentration of 1 mM, or the hydroxyl radical scavenger, dimethyl sulfoxide (DMSO) at a concentration of 5 mM for 30 minutes prior to the addition of cisplatin at a concentration of 500 μg/ml. A subsequent incubation of 5 hours is then allowed to occur. LLC-WRC 256 cells are also incubated with HBSS, DFO (1 mM) or DMSO (5 mM) for 5 hours without cisplatin as controls. One advantage of the inhibitors of the present invention, as shown in FIG. 2 for DFO and DMSO, is that CP still functions as an antineoplastic compound.

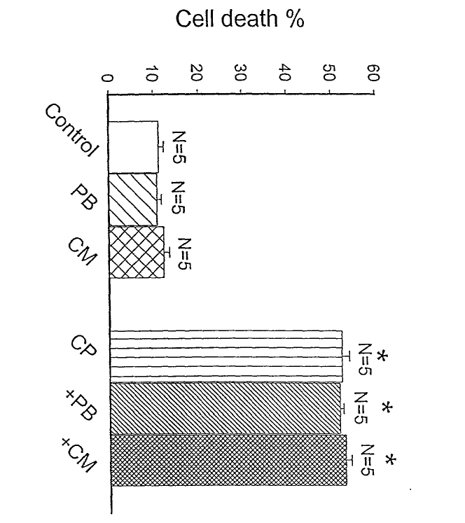

Referring now to FIG. 3 the effects of CYP inhibitors of the invention on the antineoplastic efficacy of cisplatin as measured by trypan blue exclusion are shown. A confluent LLC-WRC 256 cell monolayer is preincubated with either of the CYP inhibitors, cimetidine (CM) at a concentration of 2 mM, or piperonyl butoxide (PB) at a concentration of 1 mM for 30 minutes prior to the addition of cisplatin at a concentration of 500 μg/ml. A subsequent 5 hour incubation is then allowed to occur. LLC-WRC 256 cells are also incubated with HBSS, CM (2 mM) or PB (1 mM) for 5 hours, without cisplatin, as controls.

As evident from FIG. 3, pre-incubation of the cells with inhibitors of the invention, shown here are the iron chelator deferoxamine (DFO), the hydroxyl radical scavenger dimethyl sulfoxide (DMSO), and the CYP inhibitors cimetidine (CM) and piperonyl butoxide (PB) does not reduce the antineoplastic efficacy of CP.

Thus, as shown in FIG. 3 for CM and PB, at least one advantage of the inhibitors of the present invention, is that the inhibitors do not effect the antineoplastic activity of CP in LLC-WRC 256 cells.

Inhibitors of the present invention effectively inhibit cisplatin-induced nephrotoxicity, yet do no inhibit antineoplastic activity of cisplatin. Shown in FIGS. 2 and 3, incubation of LLC-WRC 256 cells with CP (500 μg/ml) for 2.5 hours does not increase the catalytic iron release (control 2.44±0.59; CP 2.78±0.41 nmol/mg protein, n=6) and the generation of hydroxyl radical (control 0.45±0.03; CP 0.41±0.03 nmol/mg protein, n=4) if the cells are pre-incubated with an inhibitor of the invention.

All references cited herein, including research articles, all U.S. and foreign patents and patent applications, are specifically and entirely incorporated by reference.

### EXAMPLES

The following Examples are provided merely to illustrate the present invention, and are not meant to limit the scope of the claims in any way.

### Example 1

Cell Culture

LLC-WRC 256 cells (Walker rat carcinoma cells) purchased from American Type Culture Collection (ATCC, Rockville, Maryland) were maintained in 75 cm² tissue culture flasks containing Medium 199 supplemented with 5% fetal bovine serum (Gibco, Gaithersburg, Md.) in a humidified atmosphere of 95% air-5% CO₂ at 37° C. For the experimental study, the cell monolayers were subcultured with 0.25% trypsin into a 12-well tissue culture plate until confluence. The antineoplastic efficacy of CP in LLC-WRC 256 cells was measured by the cell viability determined by the trypan blue exclusion assay. Cells failing to exclude the dye were considered non-viable, and data expressed as the percentage of non-viable cells.

### Example 2

Antineoplastic Efficacy of CP in LLC-WRC 256 Cells

Antineoplastic efficacy of CP was examined as previously described (Rawlings and Roberts, 1986, Mutation Research 166(2): 157-168.). The cells were incubated with various concentrations of CP (0-600 μg/ml) at different time points (0-6 h) in HBSS at 37° C. At the end of the incubation, the cell viability was determined.

### Example 3

Effect of Iron Chelator and Hydroxyl Radical Scavenger on the Antineoplastic Efficacy of CP

Confluent LLC-WRC 256 cell monolayer was incubated with antineoplastic dose of CP for a period of time necessary to induce consistent antineoplastic activity (CP 500 μg/ml in HBSS for 5 hours at 37° C., based on the. initial observation). The cell monolayers were preincubated with iron chelator, deferoxamine (1 mM) or hydroxyl radical scavenger, dimethyl sulfoxide (5 mM) at 37° C. for 30 minutes, washed twice with HBSS and then followed by the addition of CP.

### Example 4

Effect of CYP Inhibitors on Antineoplastic Efficacy of CP

LLC-WRC 256 cell monolayers were preincubated with cimetidine (2 mM) or piperonyl butoxide (1 mM) at 37° C. for 30 min, washed with HBSS twice and then followed by the addition of CP (500 μg/ml for 5 hours at 37° C.)

### Example 5

Effect of CP on Catalytic Iron Release

Confluent LLR-WRC 256 cells were washed twice with Chelex-treated HBSS and then incubated with a cytotoxic, dose of CP in Chelex-treated HBSS for a period of time before substantial cell killing occurred (500 μg/ml for 2.5 hours at 37° C., based on the time course study). The catalytic iron released into the medium was measured by the bleomycin assay, as described below.

### Example 6

Effect of CP on Hydroxyl Radical Formation

Confluent cell monolayers were washed with HBSS and then incubated with cytotoxic dose of CP in HBSS at 37° C. for a period of time before substantial cell killing occurred (500 μg/ml for 2.5 hours). 2-deoxy-D-ribose at a final concentration of 3 mM was added to the medium just prior to the incubation. At the end of the incubation, the medium was collected for the measurement of hydroxyl radical formation by deoxyribose degradation method as described by Halliwell et al., 1988, Meth. Biochem. Anal 33:59-90. In brief, 0.5 ml of the incubation medium was mixed with 0.5 ml of 1% (w/v). 5 thiobarbituric acid in 50 mM NaOH and 0.5 ml of 2.8% (w/v) aqueous trichloroacetic acid, heated at 100° C. for 15 min, cooled and followed by extraction with 1.5ml n-butanol. The absorbance at 532 nm of the resulting supernatant was then taken, and the hydroxyl radical level was calculated by using the extinction coefficient of 156 mM⁻¹ cm⁻¹.

### Example 7

Microsome Preparation

Microsomes from LLC-WRC 256 cells were isolated as described by Guzelian et al., 1977, Gastroenterol. 72(6): 1232-1239. Confluent cell monolayers were washed free of medium with 0.1 M potassium phosphate buffer (pH 7.4) and sonicated five times for 10 seconds each. The sonicate of the cells, and the homogenate of normal rat liver (positive control) were then centrifuged at 10,000 g for 20 minutes. The resulting supernatant was centrifuged in a Beckman L-2 ultracentrifuge at 105,000 g at 4° C. for 1 h. The microsome pellet was resuspended in 50 mM NaCl-20% glycerol (pH 7.0) (Minotti, 1989, Archiv. Biochem.Biophys 273(1): 137-143).

### Example 8

Cytochrome P450 Activity

CYP activity was measured by p-nitroanisol demethylase as described by Bissell et al., 1973, JCB 59(3): 722-734. In brief, the LLC-WRC 256 cells were sonicated. Then the sonicate and the homogenate of normal rat liver (positive control) were centrifuged at 18,000 g at 4° C. for 20 minutes. Assaying for the O-demethylase activity was carried out on the supernatant with p-nitroanisol as substrate. The reaction mixture contained 10 mM MgCl₂, 5 mM glucose-6-phosphate, 2 U/ml glucose-6-phosphate dehydrogenase, 0.4 mM p-nitroanisol, 0.5 mM NADPH, 1 mg protein/ml of the supernatant and made up with 0.1 M potassium phosphate buffer, pH 7.25, to a total volume of 1 ml. The absorbance change was. monitored at 405 to 490 nm and the CYP activity calculated using extinction coefficient of 12.87 mM⁻¹cm⁻¹.

As determined herein for LLC-WRC 256 tumor cells, a. the CYP activity (0.138±0.015 nmol/min/mg protein, n=4) and content (0.025±0.008 nmol/mg protein, n=4) were less than 10% of that in the normal rat liver (activity 2.541±0.076 nmol/min/mg protein; content 0.341±0.01 nmol/mg protein, n=8).

### Example 9

Cytochrome P450 Content

CYP content was measured by the method of Omura and Sato, 1964, JBC 239:2371-2378. In brief, the suspension of microsomes were diluted to about 1 mg of protein per ml with the assay buffer (0.1 M potassium phosphate buffer, pH 7.25, 20% glycerol, and 0.2% tergitol). After recording the baseline with a spectrophotometer, the samples were reduced with a few crystals of dithionite followed by CO bubbling for about one minute. The CO. spectrum difference of reduced microsomes was measured on a Shimadzu UV-2101 PC spectrophotometer.

### Example 10

Bleomycin Detectable Iron Assay

Iron capable of catalyzing free radical reactions (catalytic iron) was measured by the bleomycin assay as utilized in our previous studies (Baliga et al. , 1998, Kidney Int. 53:394-401, and Baliga et al., 1998, Kidney Int. 54:1562-1569). All reagents except for the experimental samples were made up in Chelex-treated pyrogen-free water and shaken with Chelex 100 to remove as much contaminating iron as possible. The reaction mixture contained in order: 0.5 ml of calf thymus DNA (1 mg/ml), 0.05 ml of bleomycin sulfate (1 mg/ml), 0.1 ml MgCl₂ (50 mM), 0.1 ml of sample, 0.1 ml of ascorbic acid (8 mM), and either HCl (50 mM) or NaOH (50 mM) to adjust pH in the range of 7.2-7.8. Sample blanks were identical expect that the bleomycin was omitted. The samples were then incubated at 37° C. for 2 hours with shaking. The reaction was stopped by adding 0.1 ml of 0.1 M EDTA, and mixed with 1 ml thiobarbituric acid (1% w/v in 50 mM NaOH) and 1 ml HCl (25%, v/v). The reaction mixture was then heated at 100° C. for 15 minutes, cooled and the resulting chromogen measured using spectrophotometer at A ₅₃₂ nm. The amount of bleomycin detectable iron in the test sample was calculated from the standard curve and the results expressed as nmol/mg cellular protein recovered from the cell monolayer.

### Example 11

Statistical Analysis

Values are expressed as mean±standard error (SE). Statistical analyses were performed using unpaired t test for only two groups and one-way ANOVA for comparisons involving more than two groups. Statistical significance was considered at p<0.01.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains. Thus, the specification and examples should be considered exemplary only with the true scope and spirit of the invention indicated by the following claims.

We claim:

1. A method for treating a patient, said method comprising the steps of:
(a) administering a therapeutically effective amount of a first composition to a patient having a neoplasia, wherein said first composition comprises an inhibitor of catalytic iron selected from the group consisting of cimetidine, piperonyl butoxide, piperine, diethyldithiocarbamate, clormethiazol, deferoxamine, dimethyl sulfoxide, and any combination thereof.

2. The method of claim 1 further comprising the step:
(b) administering a therapeutically effective amount of a second composition to the patient, wherein said second composition comprises a chemotherapeutic compound.

3. The method of claim 2 wherein said chemotherapeutic compound is cisplatin.

4. The method of claim 2, wherein step. (a) and step (b) are performed concurrently.

5. The method of claim 2, wherein said administering of step (a) is oral or parenteral, and wherein administering of step (b) is oral or parenteral.

6. A method for treating a patient, said method comprising the steps of:
(a) administering a therapeutically effective amount of a first composition to a patient having a neoplasia, wherein said first composition comprises an agent that inhibits the ability of a cytochrome P450 2E family member to serve as a source of catalytic iron.

7. The method of claim 6, further comprising the step of:
(b) administering a therapeutically effective amount of a second composition to the patient, wherein said second composition comprises a chemotherapeutic compound.

8. The method of claim 7, wherein said agent is selected from the group consisting of cimetidine, piperonyl butoxide, piperine, diethyldithiocarbamate, clormethiazol, deferoxamine, dimethyl sulfoxide, and any combination thereof.

9. The method of claim 7, wherein said chemotherapeutic compound is cisplatin.

10. A method for treating a patient, said method comprising the steps of:
(a) infecting a kidney of a patient with a vector comprising a sequence encoding an inhibitor of a cytochrome P450.

11. The method of claim 10, further comprising the step of:
(b) administering a therapeutically effective amount of a composition comprising a chemotherapeutic compound to the patient.

12. The method of claim 10, wherein said inhibitor is an antisense cytochrome P450 sequence.

13. The method of claim 12, wherein said sequence is a partial sequence of a cytochrome P450 2E family member.

14. The method of claim 11, wherein step (a) and step (b) are separated by a period of time ranging from about one minute to about one week, and wherein step (a) is carried out prior to step (b).

15. The method of claim 14, wherein said chemotherapeutic compound is cisplatin.

16. A method for treating a patient, said method comprising the steps of:
(a) infecting a kidney of a patient with a vector comprising a sequence encoding an inhibitor of a source of catalytic iron.

17. The method of claim 16 further comprising the step of:
(b) administering a therapeutically effective amount of a composition comprising a chemotherapeutic compound to the patient.

18. The method of claim 16, wherein said source of catalytic iron is a cytochrome P450 superfamily member.

19. The method of claim 18, wherein said sequence is an antisense cytochrome P450 sequence.

20. The method of claim 19, wherein said antisense cytochrome P450 sequence is SEQ.ID.NO.: 1.

21. The method of claim 17, wherein said chemotherapeutic compound is cisplatin.
